(19) United States
(12) Patent Application Publication
(10) Pub. No.: US 2023/0181517 A1
(43) Pub. Date: Jun. 15, 2023

(54) ANIMAL TREATMENT PASTES AND OINTMENTS AND METHODS OF USING SAME

(71) Applicant: Challen Ingraham, Aiken, SC (US)

(72) Inventor: Challen Ingraham, Aiken, SC (US)

(21) Appl. No.: 18/164,169

(22) Filed: Feb. 3, 2023

Related U.S. Application Data
(63) Continuation-in-part of application No. 17/503,583, filed on Oct. 18, 2021.

(51) Int. Cl. A61K 31/315; A61K 33/06; A61K 35/644; A61K 33/34; A61K 47/36; A61K 33/30; A61K 31/79; A61K 9/06

(52) U.S. Cl. CPC A61K 31/315; A61K 33/06; A61K 35/644; A61K 33/34; A61K 47/36; A61K 33/30; A61K 31/79; A61K 9/06

(57) ABSTRACT

A treatment paste includes copper sulfate, zinc oxide powder, honey, calcium chloride, and calcium hypochlorite.

### CROSS-REFERENCE TO RELATED APPLICATIONS

This application is a continuation-in-part of U.S. patent application Ser. No. 17/503,583, filed Oct. 18, 2021, the disclosure of which is hereby incorporated by reference as if fully set forth herein.

### FIELD OF THE DISCLOSURE

The present disclosure relates to antimicrobial ointments and pastes for treating certain diseases in animals. More particularly the present disclosure relates to ointments and pastes for hoofed animals having conditions, such as laminitis.

### BACKGROUND OF THE DISCLOSURE

Hoof thrush in horses and other hoofed animals has been treated mostly with liquid treatments. Thrush is a very common bacterial and/or fungal infection that occurs on the hoof of a horse or similar animal. Chlorhexidine ointments in a hydrophilic base are well-known for topically treating surface wounds in animals.

White line disease, or WLD, is characterized by an invasion of bacteria and fungi that destroy hoof-wall tissue. A crack or separation in the hoof wall, which can be caused by mechanical factors such as long toes or a club foot, may create such an opportunity for infection.

### SUMMARY OF THE DISCLOSURE

In some embodiments, a treatment paste includes copper sulfate, zinc oxide powder, honey, calcium chloride, and calcium hypochlorite.

### DETAILED DESCRIPTION

The present disclosure describes compositions for treating horses or similar animals. In one embodiment, certain ingredients as presented in Table 1 below are used to make a first “max-dose” formula, their ratios being provided as exemplary, but non-limiting:

TABLE 1

| Component | Quantity | |
|---|---|---|
| Copper sulfate | ¼ | cup |
| Zinc oxide powder | 4 | tablespoons |
| Lecithin (emulsifier) | 2 | teaspoons |
| Guar gum (stabilizer) | ½ | teaspoon |
| Pine tar | 2 | tablespoons |
| Honey | ½ | cup |
| Betadine ® | 2 | tablespoon |

It will be understood that some of the ingredients may be optional, and that that quantity of each ingredient may be varied. In at least some examples, the method of making the composition includes mixing the zinc oxide powder, Betadine® and lecithin in the quantities shown. After sufficient mixing, the honey, copper sulfate and guar gum may be added. Mixing the ingredients may occur at room temperature and may continue until the composition changes color from green to brown to produce the finished gel-like product. The product may be packaged for sale in a squeezable tube or small container.

In another embodiment, certain ingredients are used as presented in Table 2 below to make a second formula, their ratios being provided as exemplary, but non-limiting:

TABLE 2

| Component | Quantity | |
|---|---|---|
| Copper sulfate | 2 | teaspoons |
| Zinc oxide powder | 4 | tablespoons |
| Lecithin (emulsifier) | 2 | teaspoons |
| Guar gum (stabilizer) | ½ | teaspoon |
| Pine tar | 2 | tablespoons |
| Honey | ½ | cup |
| Betadine ® | 2 | tablespoons |

In yet another embodiment, certain ingredients are used as presented in Tables 3 or 5 below to make additional formulas, their ratios being provided as exemplary, but non-limiting:

TABLE 3

| Component | Quantity | |
|---|---|---|
| Copper sulfate | ¼ | cup |
| Zinc oxide powder | ¼ | cup |
| Lecithin (emulsifier) | 2 | teaspoons |
| Guar gum (stabilizer) | 1 | teaspoon |
| Pine tar | ¼ | cup |
| Honey | ½ | cup |

TABLE 4

| Component | Quantity | |
|---|---|---|
| Copper sulfate | 2 | teaspoons |
| Zinc oxide powder | ¼ | cup |
| Lecithin (emulsifier) | 2 | teaspoons |
| Guar gum (stabilizer) | 1 | teaspoon |
| Pine tar | ¼ | cup |
| Honey | ½ | honey |

TABLE 5

| Component | Quantity | |
|---|---|---|
| Copper sulfate | 2 | teaspoons |
| Zinc oxide powder | ¼ | cup |
| Honey | ½ | honey |
| Lecithin (emulsifier) | 2 | teaspoons |
| Guar gum (stabilizer) | 1 | teaspoon |
| Chlorhexadine | ½ | cup |
| Calcium chloride | 2 | tablespoons |
| Calcium hypochlorite | 2 | teaspoons |

TABLE 6

| Component | Quantity | |
|---|---|---|
| Copper sulfate | 2 | teaspoons |
| Zinc oxide powder | ¼ | cup |
| Honey | ½ | honey |
| Lecithin (emulsifier) | 2 | teaspoons |
| Guar gum (stabilizer) | 1 | teaspoon |
| Chlorhexadine | ½ | cup |
| Calcium chloride | 1 | tablespoon |
| Calcium hypochlorite | ½ | teaspoon |

In some examples, the user may apply a compound, such as that of Table 5 for a period of 2-4 days, the topical application being applied once daily to the affected area. This may be followed by topical application of the compound of Table 6 once every three days for a period of two weeks, followed by an application of the compound of Table 6 once every four days. A treatment plan may extend for between 2 and 4 weeks.

It will be understood that some of the ingredients may be optional, and that that quantity of each ingredient may be varied. In at least some examples, the method of making the composition includes mixing the zinc oxide powder, Betadine® and lecithin in the quantities shown. After sufficient mixing, the honey, copper sulfate and guar gum may be added. In this example, substantially less copper sulfate is used than in the first “max-dose” formula. Mixing the ingredients may occur at room temperature and may continue until the composition changes color from green to brown to produce the finished gel-like product. In some examples, after mixing, the ingredients may be heated to 180° then stirred until the composition cools down. In some examples, approximately 5 minutes of heating causes an exothermic reaction with the zinc and copper sulfate that results in a chemical change which creates zinc sulfate. The product may be packaged for sale in a squeezable tube or small container.

In some examples, the composition may be applied as a preventative measure. In some examples, the composition is applied every other day for two weeks. Alternatively, the composition may be applied regularly with no currently known side effects.

Although the invention herein has been described with reference to particular embodiments, it is to be understood that these embodiments are merely illustrative of the principles and applications of the present invention. It is therefore so be understood that numerous modifications may made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A treatment paste comprising:
copper sulfate;
zinc oxide powder;
honey;
calcium chloride; and
calcium hypochloiite.

2. The treatment paste of claim 1, further comprising an emulsifier that comprises lecithin.

3. The treatment paste of claim 1, further comprising a stabilizer.

4. The treatment paste of claim 1, wherein the stabilizer comprises a vegetable-based stabilizer or guar gum.

5. The treatment paste of claim 1, wherein the antiseptic solution comprises Betadine.

6. The treatment past of claim 1, further comprising honey.
